(19) United States
(12) Patent Application Publication
(10) Pub. No.: US 2025/0001036 A1
(43) Pub. Date: Jan. 2, 2025

(54) AIR CONDITIONING EQUIPMENT

(71) Applicant: NIPPON TELEGRAPH AND TELEPHONE CORPORATION, Tokyo (JP)

(72) Inventors: Asuka MIYAKE, Musashino-shi, Tokyo (JP); Hitoshi SISHIMO, Musashino-shi, Tokyo (JP); Ryuji YAMAMOTO, Musashino-shi, Tokyo (JP); Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP); Nobutomo HANZAWA, Musashino-shi, Tokyo (JP)

(73) Assignee: NIPPON TELEGRAPH AND TELEPHONE CORPORATION, Tokyo (JP)

(21) Appl. No.: 18/695,310

(22) PCT Filed: Sep. 30, 2021
(86) PCT No.: PCT/JP2021/036060

§ 371 Date: Mar. 25, 2024

(51) Int. Cl. A61L 9/20; F24F 8/167; F24F 8/22

(52) U.S. Cl. CPC A61L 9/205; F24F 8/167; F24F 8/22; A61L 2209/12; A61L 2209/14; A61L 2209/16

(57) ABSTRACT

An object of the present invention is to provide an air conditioner unit that can fully demonstrate the sterilization effect on air with a large flow rate, even by a method of irradiating a filter and a coil with ultraviolet light. The air conditioner unit according to the present invention includes a filter 20 through which air passes, a plurality of optical fibers 16 arranged in such a manner that one ends thereof are close to a surface of the filter 20 and arranged at predetermined intervals, and a light source 30 that supplies ultraviolet light from the other end of each of the optical fibers 16. Since the intensity of the light is inversely proportional to the square of the distance from the light source, if the same sterilization effect is to be obtained, the shorter the irradiation distance, the smaller the irradiation intensity of the ultraviolet light can be. That is, by bringing the tips of the optical fibers close to the surface of the filter, the filter can be sterilized by an LED having a small output.

### TECHNICAL FIELD

The present disclosure relates to an air conditioner unit for decontaminating air in an air duct.

### BACKGROUND ART

For the purpose of preventing infectious diseases, there is an increasing demand for systems for sterilization and inactivation of viruses using ultraviolet light. In the present embodiment, “decontamination” is assumed to include sterilization and virus inactivation.

An air conditioner unit installed in a building is disclosed in NPL 1. The unit performs sterilization in the air by irradiating a filter through which the air passes with ultraviolet rays. Examples of an ultraviolet light source used includes a UV lamp and an LED.

### CITATION LIST

### Non Patent Literature

[NPL 1] Mitsubishi Estate Home Co., Ltd., Website, New Aerotech, https://www.mitsubishi-home.com/lp/aerotechUV2020/(searched on Apr. 6, 2021)

### SUMMARY OF INVENTION

### Technical Problem

Air conditioner units for air sterilization mainly uses UV lamps or LEDs to radiate ultraviolet light onto internal filters and coils. The larger the air conditioner unit, the larger the air flow rate. The method of irradiating filters and coils with ultraviolet light has a problem that it is difficult to fully demonstrate the sterilization effect on air with a large flow rate.

Therefore, in order to solve the foregoing problem, an object of the present invention is to provide an air conditioner unit that can fully demonstrate the sterilization effect on air with a large flow rate, even by a method of irradiating a filter and a coil with ultraviolet light.

### Solution to Problem

In order to achieve the above-mentioned object, the air conditioner unit according to the present invention is configured to propagate ultraviolet light to a filter and a coil by an optical fiber and supply ultraviolet light to the optical fiber from the outside. In the following description, it is assumed that the “filter” includes a coil.

Specifically, the air conditioner unit according to the present invention includes a filter through which air passes, an optical fiber that irradiates a surface of the filter with ultraviolet light, and a light source that supplies the ultraviolet light to one end of the optical fiber.

Here, there are a plurality of the optical fibers which are arranged in such a manner that the other ends thereof are close to the surface of the filter and arranged at predetermined intervals. The ultraviolet light may be emitted from each of these other ends (end irradiation type).

The optical fibers may be arranged on the surface of the filter and emit the ultraviolet light from side faces thereof (side irradiation type).

The light source of the air conditioner unit according to an invention is a light emitting diode (LED).

Since the intensity of the light is inversely proportional to the square of the distance from the light source, if the same sterilization effect is to be obtained, the irradiation intensity of the ultraviolet light can be reduced when the irradiation distance is shorter. In other words, the filter can be sterilized by the LED having a small output by bringing the tips or side faces of the optical fibers close to the filter surface. For example, even a UV-LED with a current maximum output of approximately 0.1 w is theoretically sufficient to achieve a sterilization effect. With an LED light source, the replacement time can be extended from several years to several decades compared to UV lamps, reducing running costs.

Further, since the diameter of the optical fibers is approximately several hundred μm, the tips of the optical fibers can be arranged at any place on the surface of the filter, or the optical fibers can be laid. By arranging a plurality of optical fibers so that the entire filter can be irradiated with ultraviolet light, the entire filter can be sterilized.

Accordingly, the present invention can provide an air conditioner unit that can fully demonstrate the sterilization effect on air with a large flow rate, even by a method of irradiating a filter and a coil with ultraviolet light.

The filter of the air conditioner unit according to an invention has a photocatalytic layer on a surface irradiated with the ultraviolet light. The photocatalytic layer is irradiated with the ultraviolet light to generate a photocatalytic reaction, and the decontamination effect can be enhanced.

### Advantageous Effects of Invention

The present invention can provide an air conditioner unit that can fully demonstrate the sterilization effect on air with a large flow rate, even by a method of irradiating a filter and a coil with ultraviolet light.

### BRIEF DESCRIPTION OF DRAWINGS

FIG. 1 is a diagram for explaining an air conditioner unit according to the present invention.

FIG. 2 is a diagram for explaining the air conditioner unit according to the present invention.

FIG. 3 is a diagram for explaining the air conditioner unit according to the present invention.

FIG. 4 is a diagram for explaining the air conditioner unit according to the present invention.

### DESCRIPTION OF EMBODIMENTS

Embodiments of the present invention will be described with reference to the accompanying drawings. The embodiments described below are examples of the present invention, and the present invention is not limited to the following embodiments. Note that, in the present specification and the drawings, components having the same reference numerals indicate the same components.

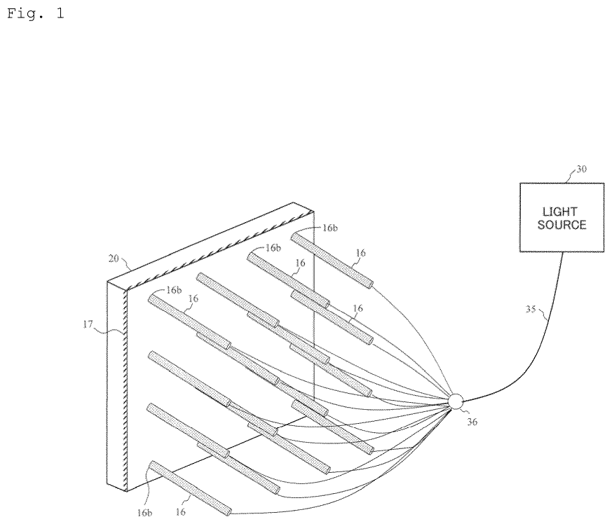

FIG. 1 is a diagram for explaining an air conditioner unit according to the present embodiment. This air conditioner unit includes a filter 20 through which air passes, a plurality of optical fibers 16 arranged so that end portions 16b are close to the surface of the filter 20, the end portions 16b being arranged at predetermined intervals, and a light source 30 that supplies ultraviolet light from the other end of each of the optical fibers 16.

The optical fibers 16 are of the end irradiation type. The end portions 16b of the tips of the optical fibers 16 are brought close to the surface of the filter 20. The end portions 16b are preferably processed into a shape (for example, uneven processing) for scattering the ultraviolet light in a plurality of directions. A plurality of optical fibers 16 are provided, and the optical fibers 16 are arranged so as to be able to cover the whole surface of the filter 20 in the region of the ultraviolet light radiated from the tips of the optical fibers 16. FIG. 1 shows an embodiment in which the surface of the filter 20 is irradiated with sixteen optical fibers, but the number and arrangement of the optical fibers 16 are not limited to those shown in FIG. 1. If the entire surface of the filter 20 can be covered with the region of the ultraviolet light radiated from the tips of the optical fibers 16, the number and arrangement of optical fibers 16 may be arbitrary.

The light source 30 is an LED that outputs ultraviolet light. The ultraviolet light that is output from the light source 30 is branched by an optical branching device 36 through an optical transmission line 35 and supplied to each optical fiber 16.

As described above, since the intensity of the light is inversely proportional to the square of the distance from the light source (tips of the optical fibers), if the same sterilization effect is to be obtained, the shorter the irradiation distance, the smaller the irradiation intensity of the ultraviolet light can be. In other words, since a plurality of optical fibers 16 are arranged on the filter surface as shown in FIG. 1, the filter 20 can be sterilized by the ultraviolet light of the LED having a small output from the light source 30.

The filter 20 preferably has a photocatalytic layer 17 on the surface irradiated with the ultraviolet light. The photocatalytic layer 17 is, for example, titanium oxide and is coated on one surface of the filter 20. The photocatalytic layer 17 generates two types of active oxygen, O₂⁻ (superoxide ion) and —OH (hydroxyl radical), by a photocatalytic reaction by being irradiated with the ultraviolet light from the tips of the optical fibers 16, and the active oxygen decomposes organic matter in the air or of the filter 20 to perform decontamination.

### EXAMPLE

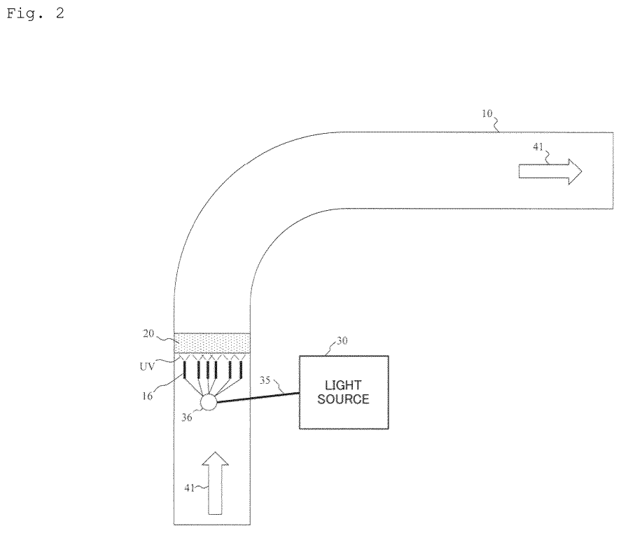

FIG. 2 is a diagram for explaining the air conditioner unit of the present embodiment installed in an air duct 10. Air is fed into the air duct 10 by a pump, not shown, and a constant air stream 41 is generated. The air stream 41 is cleaned by passing through the filter 20. Dust, debris, bacteria, viruses, and the like contained in the air stream 41 are adsorbed by the filter 20.

In this air conditioner unit, the ultraviolet light from the light source 30 is supplied to the plurality of optical fibers 16 through the transmission line 35 and the optical branching device 36. The tips of the optical fibers 16 irradiate the surface of the filter 20 with the ultraviolet light. The bacteria and viruses adsorbed by the filter 20 are decontaminated by the ultraviolet light. Note that the photocatalytic layer 17 may be present on the surface of the filter 20, as described above.

### Embodiment 2

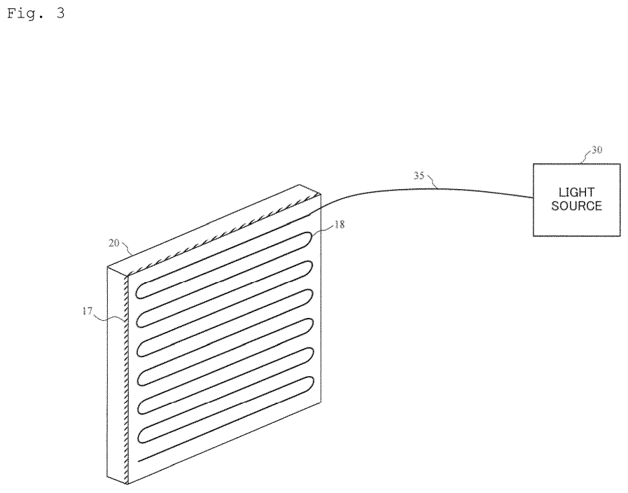

FIG. 3 is a diagram for explaining the air conditioner unit according to the present embodiment. The air conditioner unit includes the filter 20 through which air passes, an optical fiber 18 that irradiates a surface of the filter 20 with ultraviolet light, and the light source 30 that supplies the ultraviolet light to one end of the optical fiber 18, wherein the optical fiber 18 is arranged on the surface of the filter 20 and emits the ultraviolet light from a side face thereof.

The present embodiment describes only parts that differ from Embodiment 1. The optical fiber 18 is a side irradiation-type optical fiber. In FIG. 3, only one optical fiber 18 is provided, but a plurality of the optical fibers 18 may be provided by installing an optical branching device in the middle of the transmission line 35. The optical fiber 18 is arranged so as to be able to cover the whole surface of the filter 20 in the region of the ultraviolet light radiated from the side face of the optical fiber 18. Although the optical fiber 18 is arranged in a zigzag manner in FIG. 3, the arrangement of the optical fiber 18 is not limited to the one shown in FIG. 3. If the entire surface of the filter 20 can be covered with the region of the ultraviolet light radiated from the side face of the optical fiber 18, the number and arrangement of optical fibers 18 may be arbitrary.

As described above, since the intensity of the light is inversely proportional to the square of the distance from the light source (the side face of the optical fiber), if the same sterilization effect is to be obtained, the shorter the irradiation distance, the smaller the irradiation intensity of the ultraviolet light can be. That is, since the optical fiber 18 is arranged to creep over the filter surface as shown in FIG. 3, the filter 20 can be sterilized by the ultraviolet light of the LED having a small output from the light source 30.

### Example

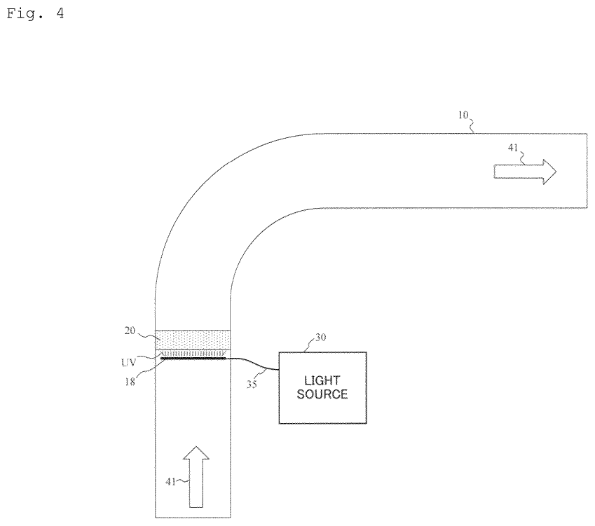

FIG. 4 is a diagram for explaining the air conditioner unit of the present embodiment installed in the air duct 10. In this air conditioner unit, ultraviolet light from the light source 30 is supplied to the optical fiber 18 through the transmission line 35. The optical fiber 18 irradiates the surface of the filter 20 with ultraviolet light from the side face thereof. The bacteria and viruses adsorbed by the filter 20 are decontaminated by the ultraviolet light. Note that the photocatalytic layer 17 may be present on the surface of the filter 20, as described above.

### (Effects)

The air conditioner unit according to the present invention has the following effects.
By installing an optical fiber on a filter surface, a sufficient decontamination effect can be obtained even by an LED light source.
An LED light source uses less power than a UV lamp and can reduce running costs.
Furthermore, since an LED light source has a longer life than a UV lamp, the frequency of replacement can be reduced and the running costs can be reduced.
Moreover, by using an optical fiber having a long service life, the cost required for replacement can be reduced.
Since the optical fiber has a small diameter and can be bent, the degree of flexibility in designing and arranging the unit can be increased.

### REFERENCE SIGNS LIST

10 Air duct
16 Optical fiber (end irradiation type)
17 Photocatalytic layer
18 Optical fiber (side irradiation type)
20 Filter
30 Light source
35 Optical transmission line
36 Optical branching device
41 Air stream

1. An air conditioner unit, comprising:
a filter through which air passes;
an optical fiber that irradiates a surface of the filter with ultraviolet light; and
a light source that supplies the ultraviolet light to one end of the optical fiber.

2. The air conditioner unit according to claim 1, wherein there are a plurality of the optical fibers which are arranged in such a manner that other ends thereof are close to the surface of the filter and arranged at predetermined intervals, and the ultraviolet light is emitted from each of the other ends.

3. The air conditioner unit according to claim 1, wherein the optical fiber is arranged on the surface of the filter and emits the ultraviolet light from a side face thereof.

4. The air conditioner unit according to claim 1, wherein the light source is a light emitting diode (LED).

5. The air conditioner unit according to claim 1, wherein the filter includes a photocatalytic layer on a surface thereof irradiated with the ultraviolet light.
